(19) United States
(12) Patent Application Publication
(10) Pub. No.: US 2020/0093452 A1
(43) Pub. Date: Mar. 26, 2020

(54) IMAGE-ASSISTED AUTOMATIC PATIENT POSITINING FOR IMPROVED IMAGING PERFORMANCE

(71) Applicant: KONINKLIJKE PHILIPS N.V., EINDHOVEN (NL)

(72) Inventors: Chuanyong BAI, SOLON, OH (US); Andriy ANDREYEV, WILLOUGHBY HILLS, OH (US); Yanfei MAO, HIGHLAND HEIGHTS, OH (US); Bin ZHANG, CLEVELAND, OH (US); Xiyun SONG, CUPERTINO, CA (US); Jinghan YE, LIVERMORE, CA (US); Shekhar DWIVEDI, WILLOUGHBY HILLS, OH (US); Zhiqiang HU, TWINSBURG, OH (US)

(21) Appl. No.: 16/471,655

(22) PCT Filed: Dec. 20, 2017
(86) PCT No.: PCT/EP2017/083679

§ 371 Date: Jun. 20, 2019

Related U.S. Application Data
(60) Provisional application No. 62/437,069, filed on Dec. 21, 2016.

(51) Int. Cl. A61B 6/00; A61B 6/03; A61B 6/04

(52) U.S. Cl. CPC A61B 6/488; A61B 6/032; A61B 6/0457; A61B 6/4417; A61B 5/055; A61B 6/469; A61B 6/465; A61B 6/545; A61B 6/037

(57) ABSTRACT

An imaging apparatus includes a nuclear medicine imaging device (10), a patient table (14), and a table controller (18) comprising an electronic processor and actuators configured to position the patient table along an axial direction and in a transverse plane that is transverse to the axial direction. An automatic positioning engine (40) comprises an electronic processor (42) programmed to determine an optimal position of the patient table in the transverse plane for imaging a target of interest in a patient based on a prior image (20, 34) of the patient. The table controller operates the patient table to position the patient table in accord with the determined optimal position of the patient table.

### FIELD

The following relates generally to the nuclear medicine imaging arts, to the positron emission tomography (PET) imaging arts, to the single photon emission computed tomography (SPECT) imaging arts, and related arts.

### BACKGROUND

So-called “hybrid” scanners that combine nuclear medicine imaging such as positron emission tomography (PET) or single photon emission computed tomography (SPECT) with transmission computed tomography (CT) imaging. These PET/CT or SPECT/CT imaging devices provide a synergistic combination of structural imaging from the CT and functional imaging from the PET or SPECT. In such studies, the PET or SPECT provides functional information so as to identify malignant tumors, necrotized tissue, or so forth, while the CT provides the structural context to locate these functional regions in the body. The CT image can also be used to generate a radiation attenuation map for use in improved reconstruction of the PET or SPECT images. The common patient transport of the PET/CT or SPECT/CT scanner facilitates spatially registering the nuclear medicine images and the CT images, and by acquiring both sets of images in a single imaging session patient changes due to weight gain or loss, bladder volume, or so forth are minimized.

In such imaging studies, the patient is usually placed in a supine position, that is, the patient lies on his or her back. This provides a stable position for the patient, limits claustrophobia by allowing the patient to look upwards, and allows for horizontal transport of the patient into the (relatively) small-diameter scanner bore. For some types of imaging, such as breast imaging, a prone position (i.e. patient lying face-down) may be employed, e.g. with the breasts positioned in conformal supports. In either the supine or prone position, the axial anatomical direction of the patient is substantially aligned with the common cylinder axis of the coaxially aligned PET (or SPECT) and CT bores. An automated patient table provides vertical adjustment that, together with centered placement of the patient on the patient table, enables the patient to be roughly centered in the plane transverse to the common bore axis. For the nuclear medicine imaging, the axial plane centered on the tumor or other internal target of interest is marked using fiduciary markers, a laser positioning system, or the like, and the automated patient table then advances the patient in the axial direction (i.e. along the common bore axis) into the imaging bore to align the central axial plane intersecting the internal target of interest at the center of the bore. In the case of PET imaging, the patient position in the central axial plane oriented transverse to the axial direction (referred to herein as the transverse plane) is generally not adjusted, as the transverse field-of-view (FOV) of the PET scanner is generally large enough to ensure the internal target of interest lies within the transverse FOV. In the case of SPECT imaging, the gamma camera used to acquire the SPECT imaging data is initially operated in a projection mode (providing a p-scope view, that is, without rotating the camera heads, or rotating over a 180° fast scan) to position each gamma camera head close to the patient in the transverse plane. Spatial registration of the resulting nuclear medicine images with the CT images is simplified by the common frame of reference provided by the common patient table.

The following discloses a new and improved systems and methods.

### SUMMARY

In one disclosed aspect, an apparatus includes a nuclear medicine imaging device, a patient table, a table controller comprising an electronic processor and actuators configured to position the patient table along an axial direction and in a transverse plane that is transverse to the axial direction, and an automatic positioning engine comprising an electronic processor. The automatic positioning engine is programmed to determine an optimal position of the patient table in the transverse plane for imaging a target of interest in a patient based on a prior image of the patient. The table controller is configured to operate the patient table to position the patient table in accord with the determined optimal position of the patient table.

In another disclosed aspect, a non-transitory storage medium stores instructions readable and executable by an electronic processing device to determine an optimal position of a patient carried by a patient table in a nuclear medicine imaging device. A prior image is retrieved. An optimal position of the patient table in the transverse plane is determined for imaging a target of interest in a patient based on the prior image of the patient. A table controller comprising an electronic processor and actuators is caused to position the patient table in accord with the determined optimal position of the patient table in the transverse plane.

In another disclosed aspect, a non-transitory storage medium stores instructions readable and executable by an electronic processing device to determine an optimal position of a patient carried by a patient table in a nuclear medicine imaging device. The stored instructions include at least the following: stored instructions readable and executable by the electronic processing device to display a graphical user interface (GUI) on a display querying whether a current nuclear medicine imaging session is a new session or a follow up session; stored instructions readable and executable by the electronic processing device to, responsive to the GUI eliciting a response indicating a new session, determine the optimal position of the patient table to locate a target of interest in the patient at a center of a transverse field of view; and stored instructions readable and executable by the electronic processing device to, responsive to the GUI eliciting a response indicating a follow-up session, determine the optimal position of the patient table to align with a prior image acquired by the nuclear medicine imaging device.

One advantage resides in providing nuclear medicine imaging with improved spatial resolution in the transverse plane.

Another advantage resides in providing nuclear medicine imaging with improved uniformity between imaging sessions.

Another advantage resides in providing nuclear medicine imaging with reduced operator-attributable variability.

Another advantage resides in providing nuclear medicine imaging for follow-up imaging sessions improved comparability with earlier imaging sessions.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a positron emission tomography (PET)/transmission computed tomography (CT) imaging device and associated components.

FIGS. 2 and 3 illustrate the position of a patient in the transverse plane before (FIG. 2) and after (FIG. 3) patient positioning as disclosed herein.

FIGS. 4 and 5 diagrammatically illustrate patient positioning workflow examples suitably implemented using the PET/CT imaging device of FIG. 1.

### DETAILED DESCRIPTION

As noted previously, patient positioning for nuclear medicine imaging typically includes axial alignment of the target of interest (e.g. an organ such as the heart or liver, or a specific lesion of interest). The axial direction is referred to herein as the z-direction, and corresponds to the common cylinder axis of the coaxially aligned PET (or SPECT) and CT bores, as well as to the axial anatomical direction of the prone or supine patient. In the transverse plane (referred to herein as the x-y plane, corresponding to an axial slice of the patient with the transverse plane containing the coronal and sagittal anatomical directions), the patient is typically positioned with the cross-section of the prone or supine patient approximately centered in the bore of the nuclear medicine imaging device. This is generally deemed sufficient since the transverse field of view (FOV) is large enough to image the entire transverse cross-section of the prone or supine patient.

However, it is recognized herein that such patient positioning in the transverse plane is non-optimal for many situations. One reason for this is that spatial resolution is generally not uniform across the transverse imaging FOV. Because of such non-uniform spatial resolution, the same object, e.g., a tumor, when located at different positions in the transverse FOV, may have different degrees of blurring, shape distortion, and quantitation degradation. Typically, the spatial resolution is the best in the center of the transverse FOV and degrades towards the edge of the transverse FOV. Furthermore, while the resolution is usually isotropic at the center of the transverse FOV (that is, the same in different direction at the center of the FOV, so that the point response is round and symmetric), the resolution away from the center is non-isotropic, i.e. different in different directions towards the edge of the FOV (usually with the point response elongated in the radial direction). As an illustrative example, the Philips Vereos PET/CT scanner exhibits spatial resolution at center of the transverse FOV of about 4.0 mm FWHM, which increases to 5.7 mm at 20 cm radially away from FOV center, as reported per NEMA NU-2-2012 standard.

In general, it is disclosed herein that the target of interest is preferably positioned at the center of the transverse FOV. In the case of a follow-up imaging session, it is preferable for the patient to be positioned in the transverse FOV similarly to the earlier imaging session so as to improve comparability with the earlier-acquired images. In general, the transverse FOV patient positioning can leverage images acquired earlier using another imaging modality, or images of the earlier imaging session in the case of a follow-up imaging session, optionally along with other information such as patient characteristics.

If the earlier imaging session (whether same-modality or different-modality) preceded the current imaging session by a significant time interval, there is some possibility the patient may have changed significantly in the intervening time interval. For example, a patient undergoing chemotherapy, radiation therapy or the like often experiences a significant weight loss. This can be accounted for in various ways. For example, in the case of a supine patient with the target of interest being the heart or a neighboring feature, it is recognized herein that the chest cavity generally does not change by a large amount due to weight loss—accordingly, the position of the heart or other target relative to the spine is likely to be substantially unchanged, so that the spine may serve as a common reference for positioning a height of an internal organ of the chest cavity (e.g. the heart) for the current imaging session with respect to the image of the internal organ in the earlier image.

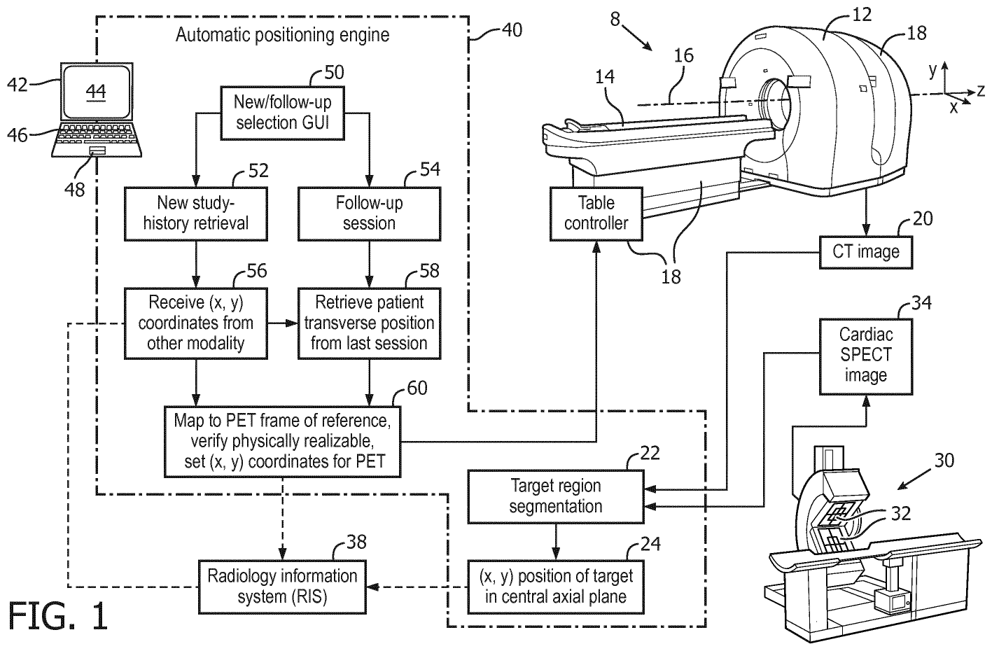

With reference to FIG. 1, an illustrative nuclear medicine imaging device comprises a hybrid system 8 including a positron emission tomography (PET) imaging device (or scanner) 10 and a transmission computed tomography (CT) imaging device (or scanner) 12. The PET/CT imaging device 8 further includes a common patient table 14 that moves a patient lying in a supine (face-up) or prone (face-down) position linearly along an axial direction 16 that corresponds to the common cylinder axis of the coaxially aligned PET (or SPECT) and CT bores of the two scanners 10, 12, as well as to the axial anatomical direction of the supine or prone patient. The z-direction parallel with the axial direction 16 is also indicated; the transverse directions x and y are also indicated, and the three directions x, y, and z are mutually orthogonal to each other. A table controller 18 comprises an electronic processor and actuators (e.g. servomechanisms with servomotors, hydraulic and/or pneumatic lifters, and the like) for providing adjustable positioning of the table 14 along the axial direction and in a transverse plane that is transverse to the axial direction. The table controller 18 is programmable to implement desired motions of the patient table 14 to move the patient axially along the z-direction as well as to move the patient height up or down along the y-direction and also in the transverse x-direction.

The disclosed patient positioning utilizes data available from previous imaging sessions. In the case of a follow-up imaging session, the previous images are acquired using the PET imaging device 10 (or, in some cases, perhaps by a different PET imaging device), from which the (x,y) position of the target of interest in the last PET imaging session is identified. For a new imaging session, the CT imaging device 12 may acquire a structural CT image 20 prior to the PET imaging. The CT image 20 is segmented in an operation 22 to identify the (x,y) position 24 of the target of interest in the CT image 20. The segmentation operation 22 may be a manual operation, e.g. using a graphical user interface (GUI) provided by a computer to enable a medical professional to delineate (i.e. contour) the target of interest, or may be an automatic or semi-automatic operation in which the contour is automatically fitted to the target of interest, for example using a mesh- or curve-fitting algorithm that fits the mesh or curve to edges of the target in the CT image 20.

With continuing reference to FIG. 1, in another typical scenario, appropriate for a cardiac patient, the patient suspected of having a cardiac malady is initially imaged by a cardiac single photon emission computed tomography (SPECT) imaging device 30 having one or more (e.g. illustrative two) gamma camera detector heads 32 to generate a cardiac SPECT image 34. Due to the relatively coarse resolution of cardiac SPECT, the SPECT image 34 may be insufficient for a cardiologist to make a definitive diagnosis, at which point the cardiologist may order a subsequent PET imaging study. To position the heart for the PET imaging, the available cardiac SPECT image 34 is segmented by the operation 22 to identify the (x,y) position 24 of the target of interest in the cardiac SPECT image 34. While CT and cardiac SPECT are illustrative “other-modality” images that may be used for identifying the (x,y) position 24 of the target of interest, any available image with sufficient information to identify the (x,y) position 24 of the target of interest may be employed, e.g. a magnetic resonance (MR) image may be employed.

In identifying the center (x,y) 24 of the target of interest in the prior image (e.g. CT image 20 or cardiac SPECT image 34) it is generally assumed that the position of the target of interest along the axial or z-direction is known. This z-position can also be derived from the prior image 20, 34, either prior to or concurrently with the operation 22, or in some embodiments the z-position may have been defined prior to acquiring these images, e.g. by a patient positioning system for the CT or cardiac SPECT imaging that aligned the target in the CT scanner 12 or cardiac SPECT device 30 prior to image acquisition.

FIG. 1 diagrammatically indicates a typical hospital information technology (IT) architecture, in which medical imaging data are stored in a Radiology Information System (RIS) 38, which stores the various images 20, 34 along with ancillary data or metadata such as the (x,y) position 24 of the target of interest. This is merely an illustrative arrangement, and other IT architectures may be employed.

For positioning the patient for the PET imaging, an automatic positioning engine 40 is implemented on an illustrative computer 42 or other electronic data processing device including an electronic processor (not shown) and, in some embodiments, user interfacing components such as an illustrative display 44 and a keyboard 46, trackpad 48, or other user input device(s). The illustrative automatic positioning engine 40 includes, or performs, the previously described target region segmentation 22, where for example the display 44 and user interfacing devices 46, 48 may enable a medical professional to contour the target of interest, and/or may display the identified (x,y) position 24 on the image 20, 34 for review/acceptance by the medical professional. It will be appreciated that the automatic positioning engine 40 may optionally be integrated with one or more other computational or electronic components, such as with the table controller 18, a controller (not shown) of the PET/CT imaging device 8, or so forth.

In a first operation, the PET scanner operator is presented with a graphical user interface (GUI) 50 via which the operator selects the current PET imaging session to be either a new PET imaging session 52 or a follow-up study 54. The GUI 50 may, for example, display radial selection buttons or check boxes on the display 44 showing the “new study” or “follow-up study” option, and the operator can select the appropriate radial button or checkbox using the available user input device(s) 46, 48.

In the case of a new study 52, the selection 52 entails identification of the other-modality image 20, 34 from which the (x,y) position of the target of interest is retrieved in an operation 56. This may be done automatically, e.g. by querying the RIS 38 to identify a most recently acquired other-modality image, such as from the CT image 20 or the cardiac SPECT image 34, or the user may be prompted to browse the patient's RIS record to identify a suitable prior other-modality study. In the case of using the CT image 20. If the (x,y) position 24 of the target of interest was previously determined and annotated to the prior other-modality image as metadata then it is read from the prior image tags or metadata in the operation 56. In an alternative process flow appropriate when such a tag or metadata is unavailable, the new study selection 52 triggers retrieval of the other-modality image 20, 34 and then triggers execution of the segmentation/center position selection operation 22 to generate the (x,y) position 24 at the time of setup of the new PET imaging study.

In the case of a follow-up study 54, the goal is not necessarily to center the target of interest at the transverse FOV center of the PET imaging device 10. Rather, the goal is to ensure the position of the subject in the transverse FOV of the PET imaging device 10 during the follow-up study is the same as in the previous PET imaging session. Accordingly, in an operation 58 the patient transverse position is retrieved from the RIS 38.

In an operation 60, the (x,y) position 24 of the target of interest, or alternatively the transverse patient position in the case of a follow-up study, is mapped to the PET frame of reference. Position limits of the table controller 18 are checked to ensure the mapped transverse position is physically realizable. The resulting position is sent to the table controller 18 which positions the patient in the specified position, after which PET imaging commences to acquire the PET images.

While described with reference to PET imaging, similar positioning approaches can be employed for SPECT imaging.

It will also be appreciated that the disclosed processing may be physically embodied as one or more non-transitory storage media (e.g. one or more hard drives, optical disks, solid state drives or other electronic digital storage devices, various combinations thereof, or so forth) that stores the instructions readable and executable by the computer 42 or other electronic data processing device including an electronic processor.

In the following, some illustrative examples are presented.

For follow-up sessions after medical interventions, the follow-up session pathway allows repeating the position of the patient of the prior treatment scan. With repeated positions, tumors/organs of interest will have the same spatial blurring, thus reducing or eliminating shape and quantitation change due to patient positioning (e.g., different resolution at different radial positions), thus allowing more reliable evaluation of the tumor/organ response to the medical interventions. For imaging organs-of-interest (OOIs) or other targets of interest, the new study pathway enables automatic positioning the OOIs at the optimized positions inside of the PET imaging FOV.

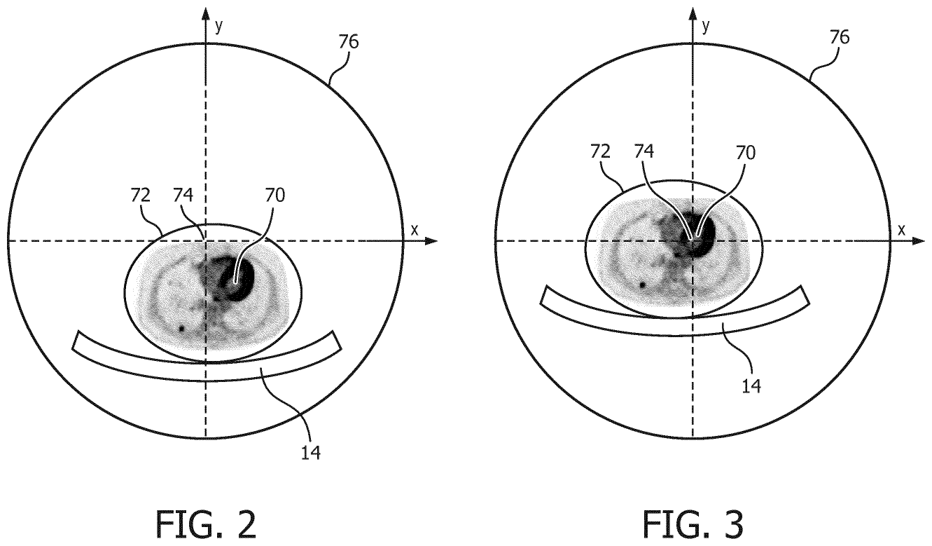

With reference to FIGS. 2 and 3, in a first illustrative example, a patient is referred to a PET myocardial perfusion (MPI) scan using the PET/CT scanner due to the ambiguous SPECT MPI results from the cardiac SPECT imaging device. The SPECT MPI image 34 used in operation 22 to automatically segment the heart 70 and the patient boundary 72. FIG. 2 illustrates the position of the heart 70 and the patient boundary 72 respective to the center 74 of the PET transverse FOV 76, assuming the patient is placed in supine (face-up) position on the subject support table 14 with the table in its standard position for PET imaging (table position in PET imaging field-of-view is hence known). The operation 60 then calculates the position of the patient in the PET scanner 10 to place the heart 70 at the center 74 of PET transverse FOV 76 while keeping the patient contour 72 inside of the FOV 76, as shown in FIG. 3. The operation 60 calculates the optimal position of the patient table 14 to control the table position during the PET scan setup or during the PET scan. By doing so, the heart 70 is closest to the center 74 of the transverse PET FOV 76, leading to the best resolution as well as most isotropic resolution in different directions. High resolution and isotropic resolution provides benefits such as sharp images of the myocardium, allowing identification of small defects; and for two, isotropic resolution eliminates/minimizes the different partial volume effect (PVE) due to anisotropic resolution, and in turn, reduces the risks of artificial under-perfusion artifacts and potential of false positive diagnosis.

To translate the other-modality image to the frame of reference of the PET imaging device 10, various approaches can be used. In one approach, suitable for a supine patient, the spine is known to be proximate to the top of the patient table 14. Thus, this provides a frame of reference for mapping the CT or SPECT image to the PET frame of reference. This approach also takes advantage of the expectation that the geometry of the spine, ribs, and contents of the chest cavity (e.g. the heart) are not expected to vary significantly even if the patient loses or gains significant weight.

In another illustrative example, if a large patient is referred to PET MPI scan while no prior images are available, CT scout view images 20 can be acquired using the CT scanner 12 without gantry rotation, ideally in two orthogonal directions, e.g. two orthogonal survey views (“surviews”). The cardiac sac is identified in the CT images 20, and the center of the heart is estimated. The patient body contour/boundary is also segmented in the CT images 20. The operation 60 calculates the best position of the heart in the PET transverse FOV while keeping the patient contour/boundary within the PET transverse FOV and calculates the table position for automatic patient positioning in PET scan.

In another illustrative example, a whole body scan with multiple targets of interest is considered, e.g. using CT or SPECT imaging. For example, the multiple targets of interest may be a cluster of tumors at different axial locations and at different transaxial locations of the patient body. The targets of interest are identified, e.g. through user input or automatic identification of the tumors in an available CT, SPECT, or other image, relative to the patient boundary, and this information is then used to calculate the optimal patient positions when scanning different axial ranges of the patient. The corresponding table positions are computed, and the table controller 18 operates the patient table 14 to position the patient in the different positions in the PET transverse FOV when scanning different axial portions of the patient. Therefore, the imaging of each of the targets is optimized. For discrete table positions, this may mean different x- and/or y-coordinates for the patient table 14 at different axial frames. For continuous bed motion, this may lead to a bed motion in both axial and transaxial directions.

In another illustrative example, for returning patients, or patients with follow-up imaging sessions on the same scanner, repeated patient positioning may be desired to minimize the image variation introduced by positioning difference. This is the follow-up session path 54 of FIG. 1. The previous PET image(s) are used to determine (calculate) the position of the patient relative to the PET transverse FOV in the previous study/studies and this information is used to calculate the position of the subject table 14 to position the patient in the same position as the previous PET imaging session. The obtained table position is sent to the table controller 18 for the desired positioning of the patient.

While PET is the illustrative nuclear medicine imaging modality for which transverse patient positioning is performed in the example of FIG. 1, the nuclear medical imaging modality could alternatively be SPECT. In this illustrative example, in the case of a follow-up SPECT and/or SPECT/CT imaging session, the positioning of the patient is calculated relative to the imaging FOV using the images from the previous imaging session. The positioning of the patient and the optimal of patient table in the FOV is then calculated. The table controller operates the table to position the patient at the desired location. For SPECT, an additional complexity is the complexity of detector head position variation during the SPECT scan. To address this, in some embodiments the detector head positions are also obtained from the previous SPECT imaging session, and the detector heads are positioned to be as close to the previous imaging session as practicable. In setting the detector head positions, the potential of patient size change between the follow-up imaging session and the previous imaging session is taken into account when determining the detector head positions during the follow-up SPECT imaging. One approach is to use a CT surview to obtain the patient boundary for the follow-up SPECT imaging session, and use the CT surview or SPECT images of the patient in the previous imaging sessions to determine the boundary of the patient. If the patient boundary at the time of the follow-up SPECT imaging session is smaller than for the previous SPECT imaging session (for example, due to patient weight loss), then the same detector head positions may be used as in the previous SPECT imaging session. If, however, the patient boundary is larger in the follow-up SPECT imaging session than the previous SPECT imaging session (e.g., due to patient weight gain), then the detector head positions are adjusted to account for the larger patient girth so as to avoid detector head-patient collisions in the follow-up imaging session.

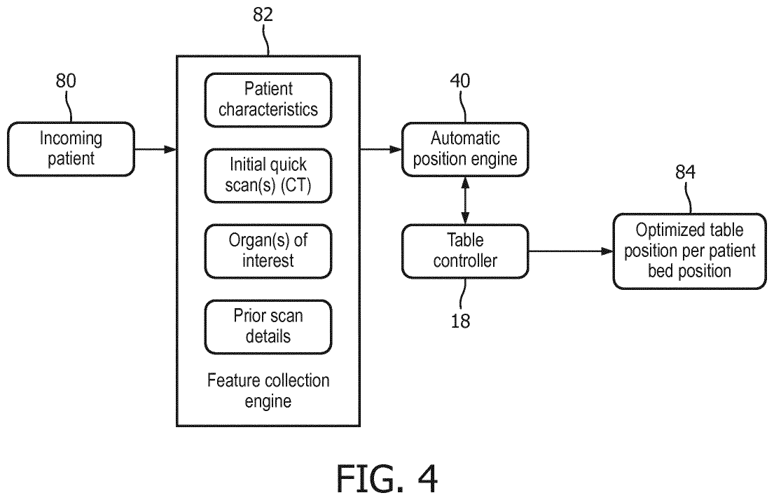

With reference to FIG. 4, an illustrative workflow for imaging using the automatic positioning engine 40 of FIG. 1 is shown. An incoming patient 80 is received for the imaging session. Various features 82 are collected for the patient, such as patient characteristics (e.g. identification, weight) and the organ(s) or other target(s) of interest to be optimally positioned, as well as previous images information such as a prior CT scan and/or prior nuclear medicine images. These features are input to the automatic positioning engine 40, along with position limits from the table controller 18, and the automatic positioning engine 40 determines the optimal transverse table position 84 of the patient table for the nuclear medicine imaging.

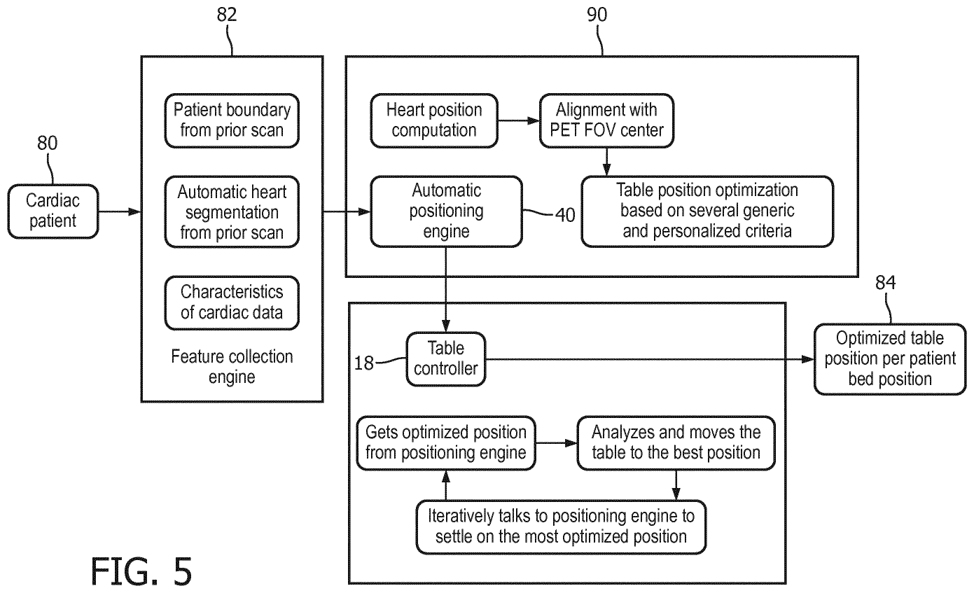

With reference to FIG. 5, a more detailed illustrative workflow for cardiac imaging is shown. As detailed in FIG. 5, in a processing block 90 the automatic positioning includes computing the heart position, aligning the heart with the center of the transverse PET FOV, and performing table optimization based on generic and patient-specific criteria. The table controller 18 in this embodiment provides feedback to the automatic positioning engine 40 in the event that the initially optimized table position is not physically realizable or is otherwise unacceptable (e.g. places part of the patient outside of the PET transverse FOV). The output is again the optimized table position for each (axial) bed position 84.

While cardiac imaging has been described as an illustrative example, the disclosed approaches are readily employed for other targets of interest besides the heart, such as for nuclear medicine imaging of a target of interest such as the heart, liver, a malignant tumor, a lung, or so forth.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An imaging device comprising:
a nuclear medicine imaging device;
a patient table;
a table controller comprising an electronic processor and actuators configured to position the patient table along an axial direction and in a transverse plane that is transverse to the axial direction; and
an automatic positioning engine comprising an electronic processor programmed to determine an optimal position of the patient table in the transverse plane for imaging a target of interest in a patient based on a prior image of the patient by operations including
segmenting the prior image of the patient to identify the target of interest and a patient boundary; and
determining the optimal position of the patient table in the transverse plane to locate the segmented target of interest at a center of a transverse field of view;
wherein the table controller is configured to operate the patient table to position the patient table in accord with the determined optimal position of the patient table.

2. (canceled)

3. The imaging device of claim 1 wherein the automatic positioning engine is programmed to determine the optimal position of the patient table in the transverse plane under a constraint that the patient boundary be inside the transverse field of view.

4. The imaging device of claim 1 wherein the automatic positioning engine is programmed to determine the optimal position of the patient table in the transverse plane under a constraint imposed by position limits of the table controller.

5. The imaging device of claim 4 wherein the patient is in a supine position, the target of interest is in a chest cavity of the supine patient, and the automatic positioning engine is programmed to determine the optimal position of the patient table in the transverse plane using a spine of the supine patient as a common reference for positioning a height of the target of interest with respect to the image of the target of interest in the prior image.

6. The imaging device of claim 4 wherein the target of interest is a heart and the prior image is a cardiac single photon emission computed tomography (SPECT) image acquired by a cardiac SPECT imaging device.

7. The imaging device of claim 1 further comprising:
a transmission computed tomography (CT) imaging device coaxially aligned with the nuclear medicine imaging device as a hybrid system with the axial direction being a common cylinder axis of the coaxially aligned nuclear medicine imaging device and CT imaging device;
wherein the prior image comprises a CT image acquired by the CT imaging device; and
wherein the automatic positioning engine is programmed to determine the optimal position of the patient table in the transverse plane using the common frame of reference of the coaxially aligned nuclear medicine imaging device and CT imaging device.

8. The imaging device of claim 1 wherein the automatic positioning engine includes a display and at least one user input device and is programmed to:
display a selection graphical user interface for receiving a selection of a new imaging session or a follow-up imaging session, wherein:
in response to receiving a selection of a new imaging session the automatic positioning engine segments the prior image of the patient to identify the target of interest and a patient boundary and determines the optimal position of the patient table in the transverse plane to locate the segmented target of interest at a center of a transverse field of view; and
in response to receiving a selection of a follow-up imaging session the automatic positioning engine determines the optimal position of the patient table in the transverse plane to align with the prior image comprising a prior image acquired by the nuclear medicine imaging device.

9. The imaging device of claim 8 wherein:
the target of interest comprises a plurality of tumors, and
the automatic positioning engine is programmed to determine an individually optimized position of the patient table in the transverse plane for imaging each tumor based on the prior image of the patient.

10. A non-transitory storage medium storing instructions readable and executable by an electronic processing device to determine an optimal position of a patient carried by a patient table in a nuclear medicine imaging device by operations including:
retrieving a prior image;
determining an optimal position of the patient table in the transverse plane for imaging a target of interest in a patient based on the prior image of the patient by operations including
segmenting the prior image of the patient to identify the target of interest and a patient boundary; and
determining the optimal position of the patient table in the transverse plane to locate the segmented target of interest at a center of a transverse field of view; and
causing a table controller comprising an electronic processor and actuators to position the patient table in accord with the determined optimal position of the patient table in the transverse plane.

11. (canceled)

12. The non-transitory storage medium of claim 11 wherein the optimal position of the patient table in the transverse plane is determined under a constraint that the patient boundary be inside the transverse field of view.

13. The non-transitory storage medium of claim 11 wherein the optimal position of the patient table in the transverse plane is determined under a constraint imposed by position limits of the table controller.

14. The non-transitory storage medium of claim 11 wherein the patient is in a supine position, the target of interest is a heart of the supine patient, and the determining uses a spine of the supine patient as a common reference for positioning a height of the heart with respect to the image of the heart in the prior image.

15. The non-transitory storage medium of claim 11 wherein the target of interest is a heart and the prior image is a cardiac single photon emission computed tomography (SPECT) image acquired by a cardiac SPECT imaging device.

16. The non-transitory storage medium of claim 11 wherein the prior image is a computed tomography (CT) image acquired by a transmission CT imaging device coaxially aligned with the nuclear medicine imaging device as a hybrid system with a common frame of reference for the nuclear medicine imaging device and CT imaging device, and the optimal position of the patient table in the transverse plane is determined using the common frame of reference.

17. The non-transitory storage medium of claim 10 wherein the prior image is a prior image of the subject acquired by the nuclear medicine imaging device and the determining comprises:
determining the optimal position of the patient table in the transverse plane to align with the prior image acquired by the nuclear medicine imaging device.

18. A non-transitory storage medium storing instructions readable and executable by an electronic processing device to determine an optimal position of a patient carried by a patient table in a nuclear medicine imaging device, the stored instructions comprising:
stored instructions readable and executable by the electronic processing device to display a graphical user interface on a display querying whether a current nuclear medicine imaging session is a new session or a follow-up session;
stored instructions readable and executable by the electronic processing device to, responsive to the GUI eliciting a response indicating a new session, determine the optimal position of the patient table to locate a target of interest in the patient at a center of a transverse field of view; and
stored instructions readable and executable by the electronic processing device to, responsive to the GUI eliciting a response indicating a follow-up session, determine the optimal position of the patient table to align with a prior image acquired by the nuclear medicine imaging device.

19. The non-transitory storage medium of claim 18 wherein the stored instructions readable and executable by the electronic processing device to determine the optimal position of the patient table to locate a target of interest in the patient at a center of a transverse field of view include:
stored instructions readable and executable by the electronic processing device to segment a prior image of the patient to identify the target of interest and a patient boundary; and
stored instructions readable and executable by the electronic processing device to determine the optimal position of the patient table in the transverse plane to locate the segmented target of interest at the center of the transverse field of view.

20. The non-transitory storage medium of claim 18 wherein the stored instructions readable and executable by the electronic processing device to determine the optimal position of the patient table to align with a prior image acquired by the nuclear medicine imaging device include stored instructions readable and executable by the electronic processing device to:
retrieve the prior image or a stored patient transverse position for the prior image;
determine the optimal position of the patient table to align with the prior image based on the prior image or the stored patient transverse position; and
causing a table controller comprising an electronic processor and actuators to position the patient table in accord with the determined optimal position of the patient table in the transverse plane.
